SEQUENCE LISTING (3 sequences)

SEQ ID NO: 1 - DNA, 18 bp, Lactobacillus acidophilus
aacgcggtgttctcggtt

SEQ ID NO: 2 - DNA, 18 bp, Lactobacillus acidophilus
cctgcactttatctatcg

SEQ ID NO: 3 - DNA, 885 bp, Lactobacillus acidophilus
gggctacacacgcgcaacaatggacggtacaacgagtcgcaagacgcgaggtttagcaaatctcttaaagccgttctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagccggtggggtaaccttttggagccagccgtctaaggtgggacagatgattggggtgaagtcgtaacaaggtagccgtaggagaacctgcggctggatcacctcctttctaaggataatttcggaaacctgcactttatctatcgtaaactttgtttagttttgagaggtttactcttaaaacatggggctttagctcagctgggagagcgcctgctttgcacgcaggaggtcaacggttcgatcccgttaaggctccattgataacattagttatcaaacttgttctttgaaaactagataatatcttttatttctttgttaattaaaataaccgagaacaccgcgttttaaagagtttaaaacattaatgtttaatcgctaaactcataaccattatcgtaagataatataggttaagttattaagggcgcatggtggatgccttggcactaggagccgatgaaggacgtgactaactgcgataagcttcggggagttgtaagtaaactgtgatccggagatttccgaatggggaaacccaacaggagtaaagacctgttatcactgagrgaatacatagctcagttaaggtagacgtggggaactgaaacatctaagtacccataggaagagaaagaaaattcgattccctgagtagtggcgagcgaa

(19) United States
(12) Patent Application Publication
(10) Pub. No.: US 2009/0203030 A1
(43) Pub. Date: Aug. 13, 2009

(54) Methods for Detecting and Quantifying Specific Probiotic Microorganisms in Animal Feed

(76) Inventors: Bryan E. Garner, Amarillo, TX (US); Matthew R. Garner, Amarillo, TX (US); Joseph F. Flint, Groton, NY (US)

Correspondence Address: ARNOLD & PORTER LLP;ATTN: IP DOCKETING DEPT., 555 TWELFTH STREET, N.W., WASHINGTON, DC, 20004-1206, US

(21) Appl. No.: 12/429,083

(22) Filed: Apr. 23, 2009

Related U.S. Application Data
(63) Continuation of application No. 10/711,155, filed on Aug. 27, 2004, now abandoned.
(60) Provisional application No. 60/481,312, filed on Aug. 29, 2003.

(51) Int. Cl. C12Q 1/68

(52) U.S. Cl. 435/6

(57) ABSTRACT

Methods and compositions are disclosed for confirming and quantifying the presence of a specific probiotic microorganism in a sample of animal feed. Hybridization and polymerase chain reaction (PCR) techniques are applied to identify the presence of the specific probiotic microorganism in cultures grown in most probable number and serial dilution methods, after calibration of the techniques using blank and control samples.

### CROSS REFERENCE TO RELATED APPLICATIONS

The present application claims the benefit of U.S. Provisional Application Ser. 60/481,312 filed Aug. 29, 2003 entitled “Method for the Detection of Microorganisms in Animal Feed,” incorporated herein by reference. The present application is related to Disclosure Document No. 529733, received Apr. 15, 2003, entitled “Analyzing Probiotics in Animal Feed”.

### FIELD OF THE INVENTION

The invention relates to materials and methods useful for the detection and quantification of specific probiotic microorganisms in animal feed. The methods include the culturing of microorganisms and use of oligonucleotide primers to detect specific probiotic microorganisms of interest.

### DESCRIPTION OF THE RELATED ART

Microorganisms are often added to animal feed in order to provide nutritional supplements, to improve digestion, to increase uptake of desirable nutrients, to compete with undesirable or harmful microorganisms, and various other reasons. Typically the microorganisms are added to the animal feed at a location where the animal feed is to be consumed by the animals, such as at a feedlot or dairy. See, for example, Ware et al. U.S. Pat. No. 5,534,271 issued Jul. 9, 1996 entitled “Process for Improving the Utilization of Feedstuffs by Ruminants,” incorporated herein by reference, and Garner et al. U.S. Pat. No. 5,529,793 issued Jun. 25, 1996 entitled “Composition for Improving the Utilization of Feedstuffs by Ruminants,” incorporated herein by reference. The terms “probiotic” and “direct fed microbials” (DFM) are often used in reference to beneficial microorganisms that are added to animal feed.

One of the challenges involved is the need to verify the presence of the added microorganisms, and to quantify their concentration. Most existing methods rely on direct or indirect culturing of samples obtained from treated feed. These methods are often compromised by the presence of other microorganisms, often in significantly higher concentrations. Additionally, many microorganisms appear similar when cultured on traditional media, further complicating their identification and quantification.

Thus, there exists a need for improved methods of analyzing treated animal feed, ideally allowing the verification of the presence of a particular strain of desired microorganism.

### SUMMARY OF INVENTION

Methods combining the culturing of samples and use of oligonucleotide primers are disclosed for the confirming and quantifying the presence of probiotic microorganisms in animal feed. The oligonucleotide primers can be used in direct detection methods, or can be used in methods such as the Polymerization Chain Reaction (PCR).

In accordance with one aspect, the invention provides a method of quantifying a presence of a specific kind of probiotic microorganism in a sample of animal feed. The method includes: (a) culturing the sample under conditions suitable for growth of cultures of the specific kind of probiotic microorganism; (b) using at least one oligonucleotide to detect the presence or absence of the specific kind of probiotic microorganism in respective portions of the cultured sample; and (c) quantifying the presence of the specific kind of probiotic microorganism in the sample of animal feed from the detected presence or absence of the specific kind of probiotic microorganism in the respective portions of the cultured sample.

In accordance with another aspect, the invention provides a method of quantifying a presence of a specific kind of probiotic microorganism in a sample of animal feed. The method includes: (a) dividing the sample into multiple portions; (b) culturing each portion of the sample under conditions suitable for growth of the specific kind of probiotic microorganism; (c) performing a polymerase chain reaction process by reacting each cultured portion of the sample successively with two oligonucleotide primers that selectively hybridize with nucleic acid of the specific kind of probiotic microorganism to produce a respective reaction product from each cultured portion of the sample; (d) detecting the presence or absence of a reaction product having a characteristic length from the reaction of each cultured portion of the sample; and (e) quantifying the presence of the specific kind of probiotic microorganism in the sample of material from the detected presence or absence of a reaction product having a characteristic length from the reaction of each cultured portion of the sample.

In accordance with yet another aspect, the invention provides a method for the detection of probiotic microorganisms in animal feed. The method includes: contacting animal feed and a probiotic microorganism to produce a treated animal feed; obtaining a sample of treated animal feed; culturing the sample under conditions suitable for growth of the probiotic microorganism; performing a polymerase chain reaction (PCR) on the cultured sample using two PCR primers to produce a PCR product; analyzing the PCR product to obtain a PCR reaction result; and correlating the PCR reaction result with the presence or absence of the probiotic microorganism in the animal feed.

In accordance with still another aspect, the invention provides a method for the detection of probiotic microorganisms in animal feed. The method includes: contacting animal feed and a probiotic microorganism to produce a treated animal feed; obtaining a sample of treated animal feed; culturing the sample under conditions suitable for growth of the probiotic microorganism to produce a culture; obtaining nucleic acid from the culture; contacting the nucleic acid with an oligonucleotide under conditions suitable for formation of a hybridized oligonucleotide-nucleic acid; detecting the hybridized oligonucleotide-nucleic acid to obtain a hybridization result; and correlating the hybridization result with the presence or absence of the probiotic microorganism in the animal feed.

### BRIEF DESCRIPTION OF SEQUENCES

The sequence listings following the detailed description below form part of the present specification and are included to further demonstrate certain aspects of the present invention. The invention may be better understood by reference to one or more of these sequences in combination with the detailed description of specific embodiments presented herein.

SEQ ID NO:1 is oligonucleotide PCR primer Lacto G5R (18 nt).

SEQ ID NO:2 is oligonucleotide PCR primer LA51 specific G4R (18 nt).

SEQ ID NO:3 is an operon ITS target rRNA sequence to which SEQ ID NO:2 hybridizes.

### BRIEF DESCRIPTION OF DRAWINGS

FIG. 1 is a summary in flow chart form of a method for quantifying the presence of a specific probiotic microorganism in a sample of animal feed.

### DETAILED DESCRIPTION

Currently used methods of verifying and quantifying the presence of desirable microorganisms added to animal feed rely solely on cultures made on petri dishes, resulting in the calculation of a “plate count” or “cfu” (colony forming unit) count. These methods are inaccurate, and cannot distinguish between similar types of microorganisms that may appear visually similar or identical when growing on a petri dish.

Various embodiments of the instant invention use oligonucleotides to either directly or indirectly detect and quantify probiotic microorganisms in animal feed.

Aspects of the instant invention relate to the use of PCR (polymerase chain reaction) methods to accurately verify and quantify the presence of probiotic microorganisms in animal feed. Other aspects of the instant invention relate to the hybridization of oligonucleotide primers to distinctive DNA or RNA sequences from one or more probiotic microorganisms of interest, followed by detection and/or quantification of the hybridized primers.

One embodiment of the invention is directed towards a method for the detection of probiotic microorganisms in animal feed, the method comprising: contacting animal feed and a probiotic microorganism to produce a treated animal feed; obtaining a sample of treated animal feed; culturing the sample in a liquid media under conditions suitable for growth of the probiotic microorganism; obtaining nucleic acid from the cultured sample; contacting an oligonucleotide with the nucleic acid under conditions suitable for hybridization; detecting the presence or absence of hybridized oligonucleotide-nucleic acid to obtain a hybridization result; and correlating the hybridization result with the presence or absence of the probiotic microorganism in the animal feed. The hybridization result can be qualitative or quantitative. The method can further comprise amplifying the nucleic acid prior to the hybridization step. The oligonucleotide can be radioactive, fluorescent, covalently bound to a reporter enzyme, or otherwise adapted to be detected. The oligonucleotide preferably hybridizes to a distinctive nucleic acid sequence present in the probiotic microorganism, but not present in other microorganisms. The nucleic acid can be DNA and/or RNA. The oligonucleotide can be DNA, RNA, PNA, or other DNA synthetic analogs.

An additional embodiment of the invention is directed towards a method for the detection of probiotic microorganisms in animal feed, the method comprising: contacting animal feed and a probiotic microorganism to produce a treated animal feed; obtaining a sample of treated animal feed; culturing the sample in a liquid media under conditions suitable for growth of the probiotic microorganism; performing a polymerase chain reaction (PCR) on the cultured sample using a first PCR primer and a second PCR primer to produce a PCR product; analyzing the PCR product to obtain a PCR reaction result; and correlating the PCR reaction result with the presence or absence of the probiotic microorganism in the animal feed.

The animal feed can generally be any type of animal feed. Examples of animal feed include dairy cattle feed, beef cattle feed, feedlot cattle, dog food, cat food, rabbit food, zoo animal food, cow feed, chicken feed, horse feed, pig feed, turkey feed, lamb feed, deer feed, buffalo feed, alligator feed, snake feed, and fish feed.

The probiotic microorganism can generally be any probiotic microorganism that is desirable to add to animal feed or to administer to an animal directly or by other means. Examples of such probiotic microorganisms include Bacillus subtilis, Bifidobacterium adolescentis, Bifidobacterium animalis, Bifidobacterium bifudum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fructosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidlactici, Pediococcus pentosaceus, Streptococcus cremoris, Streptococcus discetylactis, Streptococcus faecium, Streptococcus intermedius, Streptococcus lactis, Streptococcus thermophilus, and Escherichia coli. Another group of lactate utilizing microorganisms include Propionibacterium freudenreichii, Propionibacterium shermanii, Propionibacterium jensenii, Propionibacterium acidipropionici, Propionibacterium thoenii, Propionibacterium, Megasphaera elsdenii, Selenomonas ruminatium, and Peptostreptococcus asaccharolyticus. One specific example of a probiotic microorganism is a beneficial Lactobacillus species such as Lactobicillus acidophilus or Lactobicillus strain LA51. Strain LA51 is a naturally occurring strain. A supply of the strain LA51 has been maintained by Professor Stanley Gilliland at the University of Oklahoma, and samples have been offered under license from the University of Oklahoma.

The culturing step is preferably performed under conditions favorable for growth of the probiotic microorganism of interest. Different microorganisms have different optimal temperature, media, and pH conditions. For example, Lactobacillus acidophilus grows well in an anaerobic environment at about 35° C. and a pH of about 5.5.

The first PCR primer and second PCR primer are preferably selected to hybridize to a unique specific nucleic acid sequence present in the probiotic microorganism. The specific nucleic acid sequence is preferably not present in other microorganisms commonly found in animal feed. The specific PCR primer length and sequence depend on the nucleic acid sequence. Generally, PCR primers are about 10 nucleotides to about 25 nucleotides in length. For example, the primers can be 10, 11, 12, 13, 14, 15, 16, 17, 18, 19, 20, 21, 22, 23, 24, 25, or more nucleotides in length, up to at least 35 nucleotides. The first and second PCR primers can have the same length or can have different lengths. The PCR primers preferably do not have significant secondary structure that could interfere with hybridization to the specific nucleic acid sequence. Also, the PCR primers preferably do not have considerable repeats of sequences that may lead to false hybridization. It is also preferable that the first PCR primer and the second PCR primer do not have regions of complementarity that could lead to their hybridizing to each other rather than to the specific nucleic acid sequence.

The analyzing step can be performed by a variety of well known molecular biological methods. These methods include agarose gel electrophoresis, polyacrylamide gel electrophoresis, and liquid chromatography. These methods may include imaging techniques such as microscopic imaging of electrophoresis results.

The correlating step can include comparing animal feed samples to samples dosed with known quantities of probiotic microorganisms. The correlating step can also include comparing animal feed samples to control “blank” samples. The correlating step can be qualitative, resulting in a “yes/no” result, or quantitative, resulting in calculation of the concentration of probiotic microorganisms present in the animal feed.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Sampling of Animal Feed

An important first step in an analysis of animal feed is the obtaining of representative samples. While many suitable methods may be designed, the following has been found to be effective.

Ten samples of about 500 grams each are obtained. The samples are placed in sterile plastic bags, and are sealed. The bags are marked regarding the date and time that the sample was taken, and the amount of probiotic added to the animal feed (typically per ton of feed). Samples are obtained randomly, and from materials dispersed within the feed. For example, a 10,000 pound load of feed can be sampled once every 1,000 pounds for a total of 10 samples. If the probiotic is known or suspected of being sensitive to light, heat, or air, then the samples should be obtained from the “inside” of the feed pile.

The same number of control samples can be taken from the same type of feed that was not treated with the probiotic. The control samples are useful for determining background levels of organisms. Care should be taken with the sampling and handling equipment so as to not contaminate the control samples.

Samples can be stored in an insulated cooler, and delivered to a testing laboratory as soon as possible. The samples can be placed in a transport media, such as LBS broth, and maintained at a cool temperature, such as 4 degrees Centigrade, sufficient to inhibit growth of microorganisms, during transport to the testing laboratory.

### Example 2

### Media

Liquid or solid media should be selected to be suitable for growth of the probiotic. For example, when assaying for the probiotic Lactobacillus LA51, LBS broth and LBS agar can be used according to the manufacturer's protocols. LBS is commercially available from a wide array of suppliers including Sigma-Aldrich (St. Louis, Mo.) and Alpha Biosciences (Baltimore, Md.). LBS obtained from Alpha Biosciences has a pH of 5.5±0.2 at 25° C. and contains the following components: casein digest peptone (10.0 g/l), dextrose (20.0 g/l), yeast extract (5.0 g/l), sodium acetate (25.0 g/l), monopotassium phosphate (6.0 g/l), Tween 80 (1.0 g/l), ferrous sulfate (0.034 g/l), ammonium citrate (2.0 g/l), magnesium sulfate (0.575 g/l), manganese sulfate (0.12 g/l), and agar (for solid media, 15.0 g/l).

### Example 3

### LBS Plating of Probiotics

Ten grams of sample is added to 90 ml of 0.1% peptone in distilled water. The mixture is shaken in a mixing cylinder 30 times. The mixture is allowed to stand for 10 minutes. This is the −1 dilution.

Multiple additional serial dilutions are performed as needed to provide a reasonable number of colonies growing on an LBS plate to count. For example, dilutions of −1, −2, −3, −4, −5, and −6 can be made. Depending on the size of the plate used, a small volume of the dilution is spread evenly across the surface of the plate for culturing. Typically, 0.1 to 1 ml of liquid is used. Plates can be prepared singly or in replicates for enumeration.

Plates are covered, and incubated in an anaerobic environment for 48 hours at 35° C. The counts on the plates are determined. Typically, between 30 and 300 counts per plate is reasonable. Multiple colonies from the plate can be examined microscopically. Typically about five colonies per plate are examined. The color and shape of the colony is recorded. For the probiotic Lactobacillus LA51, the colonies should be white and round in appearance.

A slide can be prepared for a gram stain assay. LA51 colonies evaluated should be gram positive, and the organisms should appear as rounded rods.

### Example 4

### Addition of Standards to Animal Feed

Control feed is autolyzed, and allowed to cool to room temperature. The same concentration of probiotic is added to the cooled feed as was added to the treated samples. The probiotics are allowed to soak in the feed for 10 minutes. Ten grams of treated feed is added to 90 ml of 0.1% peptone in distilled water, as described in the previous Example. Serial dilutions, incubation, plating, and analysis of these samples are performed in the same manner as described in Example 3.

### Example 5

### Culturing of Probiotics from Treated Feed

2.25 liters of 0.1% peptone in distilled water is added to a mixing cylinder. Ten portions of 25 grams feed is added, one from each of the ten sample bags. A mixing ball is added, and the cylinder is shaked for 60 seconds. This is the −1 dilution. The mixture is allowed to stand for 10 minutes. Serial dilutions, incubation, plating, and analysis of these samples are performed in the same manner as described in Example 3.

### Example 6

### Culturing of Probiotics from Control Feed

The procedure from Example 3 is used with the control feed samples. This gives an indication of the background microorganisms present in untreated feed.

### Example 7

### PCR Analysis of Samples

The previous Examples can be used to obtain a “presumed” cfu count of probiotics present in animal feed. However, many organisms may appear similar or identical to the probiotic, resulting in over-counting of probiotic cfus. Also, the presence of the probiotic or other component of the animal feed treatment may stimulate or inhibit growth of non-probiotic organisms, further complicating the analysis. The use of the polymerase chain reaction (PCR) analysis technique provides clear evidence of the presence of a particular probiotic in the animal feed samples.

PCR assays for the presence (or absence) of a particular DNA sequence in a sample. PCR does not distinguish between DNA obtained from a living organism and DNA obtained from a dead or non-viable organism. Accordingly, the serial dilution cultures described in the previous Examples can be used to amplify the “signal” obtained from living organisms in the samples. The quantity of non-viable organisms would be a small percentage of the viable organisms after the incubation phase, and would therefore be of minor consequence in the subsequent PCR analysis.

PCR can be performed on specific colonies growing on plates, or on liquid culture samples. A small quantity of a colony can be added to a PCR reaction using a toothpick or the tip of a micropipette. A small volume of liquid culture (e.g. 1 microliter) can be added to the PCR reaction directly. Too much of either type of sample may inhibit the PCR reaction. A sample to be added to a PCR reaction can be centrifuged and washed in distilled water in order to eliminate fermentation products.

### Example 8

### Preparation of PCR Reaction Samples

A DNA sequence from the probiotic is selected to be amplified using PCR. Ideally, the particular DNA sequence would be unique among the microorganisms commonly found in animal feed, and would therefore act as a distinctive “marker” for the presence or absence of the probiotic in the sample. In this Example, the operon ITS target rRNA sequence was chosen (SEQ ID NO:3).

For each 25 microliter reaction, the following components are combined: 12.5 microliters HotstarTaq Master Mix (Qiagen, Inc., Valencia, Calif.), 1 microliter primer Lacto G4R (50 nanograms per microliter; 5′-MC GCG GTG TTC TCG GTT-3′ (SEQ ID NO:1)), 1 microliter primer LA51 specific (50 nanograms per microliter; 5′-CCT GCA CTT TAT CTA TCG-3′ (SEQ ID NO:2)), and 9.5 microliters distilled water. Primer SEQ ID NO:1 was chosen as a generalized sequence matching Lactobacilli. (SEQ ID NO:1 is complementary to the reverse nucleotide sequence from nucleotides 563 to 546 in SEQ NO:3.) Primer SEQ ID NO:2 is designed to hybridize to an LA51 sequence on the internal transcribed spacer (“ITS”) located between the 16S and 23S region of rRNA. (SEQ NO:2 is the nucleotide sequence from nucleotides 342 to 359 in SEQ ID NO:3.)

The sample (1 microliter liquid culture, or a small quantity of colony material) is added to the PCR reaction tube and mixed. Positive and negative control samples are also prepared.

The PCR reaction tubes are placed in a thermocycler PCR instrument, and processed using a suitable time and temperature program. For the above primers, the following program is effective: 32 cycles of (94° C. denaturing for 30 seconds, 54° C. annealing for 30 seconds, and 72° C. polymerizing for 1 minute), then 72° C. for 10 minutes, and storage at 4° C.

PCR products are readily analyzed using horizontal agarose gel electrophoresis. A 1.75% agarose gel made in 1×TAE buffer containing 0.1 microliter per ml ethidium bromide can be used. For the above described PCR reaction, 8 microliters of reaction mixture is combined with 2 microliters of 5× loading buffer (containing bromphenol blue marker), and added into a well in the agarose gel. A size standard (e.g. phiX 174 DNA cut with restriction enzyme Haelli) is added into one lane of the gel. The gel is run at 25-50 volts. Progress of the electrophoresis is monitored by visual inspection of the bromphenol blue band in the gel. DNA bands are visualized using a UV light source. PCR analysis of DNA from probiotic Lactobacillus LA51 using primers SEQ ID NOS:1 and 2 produces a single band of about 225 bp.

PCR reactions using various known concentrations of standards can be used to quantify the concentration of probiotic in the culture. This, combined with the degree of serial dilution, can be used to quantify the concentration of probiotic in the animal feed.

### Example 9

### Interpretation of Assay Results

Animal feed can be treated with probiotic Lactobacillus LA51 at 2.0×10 exp 10 to 2.6×10 exp 10 cfu/g. The following results are expected from using the methods described in the previous Examples. Most Probable Number (“MPN”) is a method for estimating low concentrations of organisms based on observation of serial dilutions (Cochran, W. G. 1950. Estimation of bacterial densities by means of the “Most Probable Number.” Biometrics 6:105-116; James T. Peeler and Foster D. McClure; Bacteriological Analytical Manual, USFDA, 7th edition, 1992).

| Sample | Plate Count | Most Probable Number |
|---|---|---|
| LA51 probiotic culture | 2.4 × 10¹⁰/g | 2.0-2.4 × 10¹⁰/g |
| Control feed | 1 × 10³/g-1 × 10⁷/g | 0 |
| Autolyzed (lab treated) | 5 × 10⁴/g-1.6 × 10⁵/g | 5 × 10⁴/g-1.6 × 10⁵/g |
| Treated feed | 5 × 10⁴/g-1.6 × 10⁷/g | 5 × 10⁴/g-1.6 × 10⁵/g |

Control feeds containing LA51 are most likely contaminated.

### Example 10

### Exemplary Assay Results

Animal feed was treated with probiotic Lactobacillus LA51 at 2.0×10exp10 cfu/g. The probiotic was allowed to contact the feed for 5.5 hours prior to sampling. The following counts were determined, and were all found to be within the expected ranges.

| | Background | Detected LA51 | Expected LA51 |
|---|---|---|---|
| Culture | 0 | 2.4 × 10¹⁰ | 2.0 × 10¹⁰ |
| Control feed | 3.7 × 10⁵ | 0 | 0 |
| Autolyzed/treated | 0 | 7.3 × 10⁴ | 1.0 × 10⁵ |
| Treated feed | 3.7 × 10⁵ | 6.7 × 10⁴ | 1.0 × 10⁵ |

Next, samples were observed using a microscope and by gram staining

| | Colonies observed | Microscopic | Gram Stain |
|---|---|---|---|
| Culture of LA51 | 5 round white | Round rods | Gram+ |
| Control Feed | 3 irregular/clear | Cocci | Gram− |
| | 2 large white | Long rods | Gram+ |
| Autolyzed/treated | 5 round white | Round rods | Gram+ |
| Treated feed | 2 irregular/clear | Cocci | Gram− |
| | 3 round white | Round rods | Gram+ |

The Control Feed and Treated Feed contained similar levels of presumptive LA51 counts and similar observed organisms. However, by observing amplified PCR products, only the Treated Feed contained LA51. About 43 percent of expected organisms were extracted from the feed by use of a mixing ball. This allowed for positive identification of LA51, and also assured a level within the expected range of content of organisms. While only 43% of expected organisms seems to be low, obtaining 100% of expected live organisms is somewhat unrealistic. Any recovery above 10% places the determination within the same logarithm of expected counts.

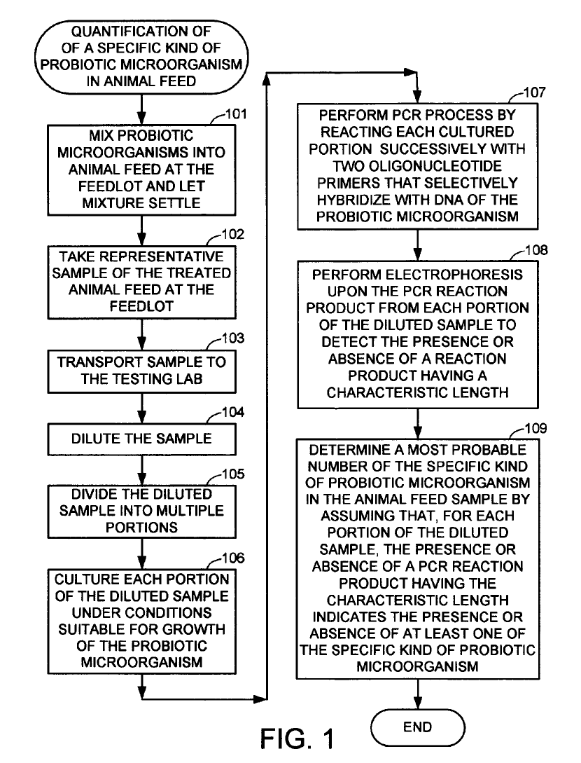

FIG. 1 shows a summary of a method employed in a number of the above examples for detecting and quantifying the presence of a specific kind of probiotic microorganism in animal feed. This method is suited for automated processing of a sample and quantification of low concentrations of a specific kind of probiotic microorganism without the use of radioactive markers or probes. In a first step 101, probiotic microorganisms are mixed into animal feed at the location where the animal fed is to be consumed by the animals, such as at a feedlot, and the mixture is allowed to settle for a certain interval of time. Then in step 102, a representative sample of the treated animal feed is taken from the feed pile at the feedlot. The sample is taken so as to be representative of the bulk of the feed to be consumed at the feedlot. In step 103, the sample is transported to the testing lab

In step 104, the sample is diluted so that in a later step (109) a good number of cultured portions of the sample will have indications of the absence of the specific probiotic microorganism of interest. Step 104 may be omitted if the initial concentration of the specific probiotic microorganism is sufficiently low.

In step 105, the diluted sample is divided into multiple portions. In step 106, each portion of the diluted sample is cultured under conditions suitable for growth of the probiotic microorganism. In step 105, a PCR process is performed by successively reacting each cultured portion with two oligonucleotide primers that selectively hybridize with DNA of the probiotic microorganism.

The number of PCR amplification cycles to be used upon each cultured portion can be chosen by preparing standard samples each containing a small number of the probiotic microorganism per sample, and performing PCR amplification upon the standard samples using respective numbers of cycles spread over a wide range of cycles. There should be a minimum number of cycles at which a positive indication is obtained (by electrophoresis detection as in step 108). There may be a maximum number of cycles at which a positive indication is no longer valid. The number of cycles to be used upon each cultured portion should be a median between these minimum and maximum numbers. This calibration of the PCR process can also be done upon standard samples prepared by adding known quantities of the probiotic microorganism to sterilized and unsterilized quantities of the animal feed, in order to adjust the number of PCR cycles to compensate for effects of the animal feed upon the probiotic microorganism or competing microorganisms in the animal feed.

In step 108, electrophoresis is performed upon the PCR reaction product from each portion of the diluted sample to detect the presence or absence of a reaction product having a characteristic length.

In step 107, the most probable number of the specific kind of probiotic microorganism in the animal feed sample is determined by assuming that, for each portion of the diluted sample, the presence or absence of a PCR reaction product having the characteristic length indicates the presence or absence of at least one of the specific kind of probiotic microorganism. The number of portions of the diluted sample indicated as having at least one of the specific kind of probiotic microorganism is a lower bound to the number of the specific kind of probiotic microorganism in the portions of the sample prior to incubation.

By assuming that the specific kind of probiotic microorganism in the sample are randomly distributed among the sample portions, one can determine the most probable number of the specific kind of probiotic microorganism initially in the sample from the number of sample portions indicated as having at least one of the specific kind of probiotic microorganism. Moreover, confidence limits can be established that also take into account random variation of the sample from the bulk of the treated animal feed from which the sample is taken.

For example, tables showing the most probable number of microorganisms and high and low 95% confidence limits given a particular number of positive indications for the cases of N=3, 5, 8, and 10 sample portions are published on the Internet web site of the Center for Food Safety & Applied Nutrition of the U.S. Federal Drug Administration (cfsan.fda.gov) in the Bacteriological Analytical Manual Online, January 2001, Appendix 2, Most Probable Number from Serial Dilutions, by Robert Blodgett. Data in the table for the case of N=10 sample portions are reproduced below:

| No. Positives | Most Probable No. | Low Conf. Limit | High Conf. Limit |
|---|---|---|---|
| 0 | <1.1 | — | 3.3 |
| 1 | 1.1 | 0.5 | 5.9 |
| 2 | 2.2 | .37 | 8.1 |
| 3 | 3.6 | .91 | 9.7 |
| 4 | 5.1 | 1.6 | 13 |
| 5 | 6.9 | 2.5 | 15 |
| 6 | 9.2 | 3.3 | 19 |
| 7 | 12 | 4.8 | 24 |
| 8 | 16 | 5.9 | 33 |
| 9 | 23 | 8.1 | 53 |
| 10 | >23 | 12 | — |

Serial dilutions can be performed in step 104, and steps 105 to 109 can be performed upon each of the dilutions in the series. A most probable number of the specific kind of probiotic microorganism in the sample can be determined for each dilution in the series from a table, and the most probable number having the best confidence limits can be selected as the most probable number of the specific kind of probiotic microorganism in the sample. Some of the tables in the above-cited Bacteriological Analytical Manual Online, January 2001, Appendix 2, also enable a most probable number to be determined based on the combination of indications from different dilutions in a series.

As discussed above, the most probable number of the specific kind of probiotic microorganism determined for the sample can be compared to the number determined for samples of known quantities of the specific probiotic microorganism and with control samples known to have none of the specific probiotic microorganism. The samples of known quantities and the control samples can confirm that the hybridization and polymerase chain reaction (PCR) techniques are in fact detecting the presence of the specific probiotic microorganism in the cultures grown in the most probable number and serial dilution methods.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

1. A method of quantifying a presence of a specific kind of probiotic microorganism in a sample of animal feed, said method comprising:
(a) culturing the sample under conditions suitable for growth of cultures of the specific kind of probiotic microorganism;
(b) using at least one oligonucleotide to detect the presence or absence of the specific kind of probiotic microorganism in respective portions of the cultured sample; and
(c) quantifying the presence of the specific kind of probiotic microorganism in the sample of material from the detected presence or absence of the specific kind of probiotic microorganism in the respective portions of the cultured sample.

2. The method as claimed in claim 1, which includes taking the sample of animal feed from a feedpile and transporting the sample to a testing lab in such a way that the sample of the animal feed at the testing laboratory is representative of the condition of the animal feed when the animal feed is to be consumed by animals.

3. The method as claimed in claim 1, which includes taking the sample of animal feed from a feedpile at a location where the animal feed is to be consumed by animals.

4. The method as claimed in claim 1, wherein the specific kind of probiotic microorganism is a species of Lactobacillus.

5. The method as claimed in claim 1, wherein the specific kind of probiotic microorganism is Lactobacillus acidophilus.

6. The method as claimed in claim 1, wherein the specific kind of probiotic microorganism is Lactobacillus LA-51.

7. The method as claimed in claim 1, wherein said at least one oglionucleotide hybridizes with a nucleic acid sequence that is indicative of a species of the specific kind of microorganism.

8. The method of claim 1, wherein the sample is cultured on a plate of culture media, and the respective portions of the cultured sample are taken from respective colonies of microorganisms that have been found to have grown on the plate of culture media.

9. The method of claim 1, wherein the sample is cultured by dividing the sample into multiple portions and culturing each portion, and wherein the presence or absence of the specific kind of microorganism is detected in each cultured portion.

10. The method as claimed in claim 9, wherein the sample is divided into the multiple portions by diluting the sample and dividing the diluted sample into the multiple portions.

11. The method as claimed in claim 9, wherein the sample is divided into multiple portions by mixing the sample with liquid to produce a fluid mixture, and dividing the fluid mixture into the multiple portions.

12. The method as claimed in claim 1, wherein the using of at least one oligonucleotide to detect the presence or absence of the specific kind of probiotic microorganism in respective portions of the cultured sample includes detecting the presence or absence of a product of hybridization of said at least one oglionucleotide with a nucleic acid sequence that is indicative of the specific kind of probiotic microorganism.

13. The method as claimed in claim 1, wherein the using of at least one oligonucleotide to detect the presence or absence of the specific kind of probiotic microorganism in respective portions of the cultured sample includes using two oligonucleotide primers that induce a polymerase chain reaction in the presence of nuclear material of the specific kind of probiotic microorganism, and detecting the presence or absence of a product of the polymerase chain reaction of the two oligonucleotide primers in the presence of the nuclear material of the specific kind of probiotic microorganism.

14. The method as claimed in claim 13, wherein one of the oglionucleotide primers hybridizes with a nucleic acid sequence indicative of the genus of the specific kind of microorganism, and another of the oglionucleotide primers hybridizes with a nucleic acid sequence indicative of the species of the specific kind of probiotic microorganism.

15. The method as claimed in claim 13, wherein the detecting of the presence or absence of a product of the polymerase chain reaction of the two oligonucleotide primers in the presence of the nuclear material of the specific kind of probiotic microorganism includes performing electrophoresis of polymerase chain reaction products to detect a reaction product having a characteristic molecular length indicative of a polymerase chain reaction of the two oligonucleotide primers in the presence of the nuclear material of the specific kind of probiotic microorganism.

16. The method as claimed in claim 1, wherein the presence of the specific kind of probiotic microorganism in the sample of material is quantified in terms of a most probable number of the specific kind of probiotic microorganism.

17. A method of quantifying a presence of a specific kind of probiotic microorganism in a sample of animal feed, said method comprising:
(a) dividing the sample into multiple portions;
(b) culturing each portion of the sample under conditions suitable for growth of a culture of the specific kind of probiotic microorganism;
(c) performing a polymerase chain reaction process by reacting each cultured portion of the sample successively with two oligonucleotide primers that selectively hybridize with nucleic acid of the specific kind of probiotic microorganism to produce a respective reaction product from each cultured portion of the sample;
(d) detecting the presence or absence of a reaction product having a characteristic length from the reaction of each cultured portion of the sample; and
(e) quantifying the presence of the specific kind of probiotic microorganism in the sample of material from the detected presence or absence of a reaction product having a characteristic length from the reaction of each cultured portion of the sample.

18. The method as claimed in claim 17, wherein the presence of the specific kind of probiotic microorganism in the sample of material is quantified in terms of a most probable number of the specific kind of probiotic microorganism in the sample of material.

19. The method as claimed in claim 17, wherein the sample is diluted prior to the culturing of the portions of the sample so that a good number of the cultured portions of the sample have an absence of a reaction product having the characteristic length.

20. The method as claimed in claim 17, wherein the two oglionucleotide primers include one oglionucleotide primer that hybridizes with a nucleic acid sequence indicative of a genus of the specific kind of probiotic microorganism, and another oglionucleotide primer that hybridizes with a nucleic acid sequence indicative of the species of the specific kind of probiotic microorganism.

21-36. (canceled)
